(19) United States
(12) Patent Application Publication
(10) Pub. No.: US 2008/0047027 A1
(43) Pub. Date: Feb. 21, 2008

(54) Activation of Nuclear Transfer Embryos

(75) Inventors: Christopher Gerald Grupen, Westbourne Park (AU); Mark Brenton Nottle, Bibaringa (AU)

Correspondence Address: FISH & RICHARDSON P.C., P.O. BOX 1022, MINNEAPOLIS, MN, 55440-1022, US

(73) Assignees: Relag Pty Ltd., Victoria (AU); Garelag Pty Ltd., Victoria (AU)

(21) Appl. No.: 11/610,988

(22) Filed: Dec. 14, 2006

Related U.S. Application Data
(63) Continuation of application No. 10/466,057, filed on Mar. 22, 2004, now abandoned.

(30) Foreign Application Priority Data
Jan. 10, 2001 (AU) PR2479

(51) Int. Cl. A01K 67/02

(52) U.S. Cl. 800/15; 800/16; 800/17

(57) ABSTRACT

Methods for the activation of nuclear transferred embryos using elevated calcium levels introduced into cells, and a maturation promoting factor (MPF) inhibitor are described. Elevated calcium levels introduced into the embryo cells from a culture medium containing elevated calcium levels, in the range 2 mM to about 12 mM, followed directly and immediately by incubation with DMAP are described. Also described are animals produced from embryos so treated.

### BACKGROUND OF THE INVENTION

The present invention relates to methods for improving the efficiency of activation of nuclear transfer embryos. The methods are applicable to all mammals, and are particularly beneficial to porcine nuclear transfer. The efficiency of activation of nuclear transfer embryos is dramatically improved according to this invention.

Nuclear transfer involves insertion of a donor cell or nucleus (karyoplast) into an enucleated oocyte (cytoplast) and reprogramming of the donor nucleus by the recipient cytoplasm. In general, nuclear transfer protocols include:
enucleation of the chromosomes from the recipient oocyte;
transfer of donor nucleus to the enucleated oocyte to give a single cell nuclear transfer (NT) embryo; and
activation of the NT embryo oocyte.

An activated single cell NT embryo is a viable embryo, capable of cell division to give a multicellular activated embryo, which is competent to develop in culture to a blastocyst stage.

Activated nuclear transfer embryos may be introduced into a pregnancy competent host uterus, for example, after culture to the blastocyst stage, to give cloned animals, genetically manipulated by standard techniques, or used in many different ways as described hereafter.

Nuclear transfer or cloning using somatic cells has been successfully performed in a variety of animals such as cattle (Cibelli et al 1998 Science 280:1256) and sheep (Wilmut et al (1997) Nature 385:810).

The prior art (see, for example, Susko-Parrish U.S. Pat. No. 6,077,710) teaches activation of unclear transfer oocytes with calcium in the presence of an ionophore, followed by prevention of phosphorylation of cellular proteins using phosphorylation inhibitors such as serine-threonine kinase inhibitors. This method has been successfully used in animals such as cattle, but does not work in pigs.

However, the efficiency with which animals can be cloned is very low (around 1% for sheep and cattle embryos constructed using somatic cells) and improvements in efficiency are greatly needed. A particular problem is the percentage of active NT embryos oocytes which develop to blastocysts, the multi cell embryo development stage at which embryos are transplanted into a pregnancy competent host uterus. The present invention addresses, at least in part, the problems of the prior art.

### SUMMARY OF THE INVENTION

The present invention relates in its broadest aspect to a method for the activation of nuclear transferred embryos, particularly porcine nuclear transfer embryo, using elevated calcium levels which may be introduced into cells by calcium ionophores or other physical or chemical means and using a Maturation Promoting Factor (MPF) inhibitor, such as 6-dimethylaminopurine (6-DMAP). Incubation of the nuclear transfer embryo with an MPF inhibitor, such as DMAP directly and immediately follows calcium induced activation. Culture of the activated embryo gives rise to activated multicellular embryos.

In another aspect the invention relates to activating a nuclear transfer embryo of a mammal, such as an ungulate comprising incubating the embryo in an embryo culture medium including an elevated calcium level and a divalent cation ionophore for a period sufficient to introduce calcium into the embryo to give a calcium loaded embryo, immediately removing the ionophore by washing the embryo in the presence of an MPF inhibitor, and thereafter incubating the calcium loaded embryo with the an MPF inhibitor to give an activated nuclear transfer embryo. Culture of the activated embryo in conventional embryonic culture medium gives rise to developmentally programmed cell division, for example, to the blastocyst stage.

The invention includes activated single cell embryos, activated multicellular embryos (for example, from 2-cells to blastocysts) and animals when produced following introduction of multicellular embryos into a pregnancy competent uterus.

Using the present method we have increased parthenogenetic activation (a model for activation of nuclear transfer embryos) from around 15% as achieved with the prior art electrical activation, to approximately 50% by the methods of the invention.

The invention is suitable for use with all well known nuclear transfer protocols including simultaneous fusion and activation, fusion before, activation post fusion enucleation, serial nuclear transfer etc.

The invention is suitable for all species, in particular ungulates, and may offer an improvement in efficiency even where prior art methods have been shown to give acceptable results, for example, cattle.

### DETAILED DESCRIPTION OF INVENTION

Nuclear transfer involves insertion of a donor cell or nucleus (karyoplast) into an enucleated oocyte (cytoplast) and reprogramming of the donor nucleus by the recipient cytoplasm. Nuclear transfer methods are well known in the art.

However, as mentioned above the efficiency with which animals can be cloned is very low (around 1% for sheep and cattle embryos constructed using somatic cells) and improvement is greatly needed.

The present invention relates in its broadest aspect to a method for the activation of nuclear transferred embryos, particularly porcine nuclear transfer embryo, using elevated calcium levels which may be introduced into cells by calcium ionophores or other physical or chemical means and using an MPF inhibitor, such as 6-dimethylaminopurine (6-DMAP). Incubation of the nuclear transfer embryo with an MPF inhibitor, such as DMAP directly follows calcium induced activation. Culture of the activated embryo gives rise to activated multicellular embryos.

In another aspect the invention relates to activating a nuclear transfer embryo of a mammal, such as an ungulate comprising incubating the embryo in an embryo culture medium including an elevated calcium level and a divalent cation ionophore for a period sufficient to introduce calcium into the embryo to give a calcium loaded embryo, immediately removing the ionophore by washing the embryo in the presence of an MPF inhibitor, and thereafter incubating the calcium loaded embryo with the an MPF inhibitor to give an activated nuclear transfer embryo. Culture of the activated embryo in conventional embryonic culture medium gives rise to developmentally programmed cell division, for example, to the blastocyst stage.

An elevated calcium level is a calcium level higher than that used for calcium induced activation of embryos in the prior art. An elevated level is generally in the range from about 2 mM Ca²⁺ to about 12 mM Ca²⁺, for example, 6 mM Ca²⁺ to 9 mM Ca²⁺.

Calcium loading of an embryo in the presence of a divalent cation ionophore is a relatively rapid step and may, for example, involve incubation in the high calcium/ionophore medium for a period of five seconds to one hour such as three minutes to ten minutes, by way of further example, five minutes. The ionophore and high calcium levels are then immediately removed by washing in the presence of the MPF inhibitor. This is an important feature of this aspect of the invention.

Calcium loaded embryos are cultured with the MPF inhibitor for a suitable time period, such as from one hour to ten hours, for example, from two hours to five hours, such as three hours at 37° C. 6-methylaminopurine (6-DMP) is an example of an MPF inhibitor. Other MPF inhibitors such as inhibitors of phosphorylation may be used.

The inventors have found that
1 Porcine nuclear transfer embryos need to be activated in media containing increased calcium concentrations than that normally used (for example, 7.7 mM compared to 1.4 mM).
2 Porcine nuclear transfer embryos need to be incubated immediately in a phosphorylation agent, such as 6-DMAP following activation with an ionophore, such as ionomycin (during ionophore wash-out). A delay of even a few minutes as is used routinely in cattle results in little or no activation.

These findings have application to other animals.

Using the present method we have increased parthenogenetic activation (a model for activation of nuclear transfer embryos) from around 15% using electrical activation to approximately 50%.

The method is suitable for use with all nuclear transfer protocols including simultaneous fusion and activation, fusion before, activation post fusion enucleation, serial nuclear transfer, etc.

The method is suitable for all species, in particular ungulates, and may offer an improvement in efficiency where this method has been shown to give acceptable results, for example, cattle.

Enucleation may be effected by various methods including bisection of the oocyte, enucleation of the metaphase plate, enucleation at telophase, enucleation at the time of pronuclear formation. Alternatively the oocyte may be allowed to self enucleate.

Transfer of the donor cell or karyoplast into the recipient cytoplast containing its recipient chromosomes may be effected by a variety of techniques. For example, membrane fusion, direct injection of the karyoplast into the recipient cytoplast, or other means to give an NT embryo. At the time of karyoplast insertion, the reconstructed embryo may be activated by physical or chemical means. Alternatively, activation may take place before or following insertion of the donor nucleus.

Donor cells can also be embryonic cells, embryonic stem cells, primary cell cultures, cultured cell lines derived from embryonic, foetal or adult somatic cells, and the like. By way of further example, an embryonic cell may be a blastomere, for example, a 16 to 32 cell mass (morula), or a pluripotent cell derived from a blastocyst. The donor cell may be subject to conventional recombinant DNA manipulation.

The donor cells may come from any animal as described previously, including livestock animals or companion animals. A donor cell derived from an animal can be isolated from nearly any type of tissue or organ.

Fibroblasts can be preferable because they are easily obtained (either from foetal or adult tissue sources), can be obtained in large quantities and are easily propagated, genetically modified and cultured in vitro.

The importance of synchronizing the cell cycle between the oocyte and the donor nucleus has been demonstrated previously. High levels of maturation promoting activity in the metaphase II oocyte result in irreversible damage to the chromatin and aneuploid following reconstruction (Campbell et al 1993 Biology of Reproduction 49:933). To overcome this problem the cell cycle of the donor nucleus needs to be in metaphase or G1 of the cell cycle. Donor nuclei can have the cell cycle synchronized using a variety of methods such as serum starvation (Wilmut et al 1997), growth to confluence (Onishi et al 2000), etc. Non-synchronized populations can also be used (Cibelli et al 1998). Alternatively the oocyte or recipient cytoplast can be activated to reduce MPF levels (so called universal recipient)

The recipient cytoplast can be an oocyte, zygote or any cell from an embryo. Suitable animal sources of oocytes can be as described above for sources of donor nuclei. Preferably, the oocytes are obtained from a vertebrate animal and more preferably, an ungulate. Ova or oocytes may be readily collected from the reproductive tracts of ovulating animals using surgical or non-surgical methods. Methods for isolating oocytes are well known in the art. Ovulation may be induced by administering gonadotropins of various species origin to animals. Oocytes may be collected by aspiration from mature follicles, or collected following ovulation. Alternatively immature oocytes may be collected from the ovaries of living or slaughtered animals and matured in vitro using standard procedures such as described in WO 90/13627 (“In vitro maturation of bovine oocytes in media containing recombinant gonadotropins along with bovine oviductal cells”, 1989). Oocytes can be fertilized in vivo or in vitro to yield zygotes.

Again, the recipient cytoplast may come from any animal as described above for donor nucleus, including livestock animals or companion animals. Preferably the donor cell and recipient cell are from the same species.

Cell fusion may be carried out by any means known in the field. Established methods for inducing cell fusion include exposure of cells to fusion-promoting chemicals, such as polyethylene glycol (see, for example, Kanka et al, (1991), Molecular Reproductive Development, 29:110-116), the use of inactivated virus, such as sendi virus (see, for example, Graham et al, (1969), Wistar Inst. Symp. Monogr., 9:19), and the use of electrical pulses (see, for example, and Prather et al, (1987), Biol. Reprod., 37:859-866).

Alternatively a donor nucleus (karyoplast) can be isolated from a cell and injected directly into the cytoplasm of the recipient cytoplast. Direct micro injection of a karyoplast into a donor cell may be carried out by conventional method, such as disclosed by Wakayama et al (1998) Nature 394:369).

Betthauser et al (2000, Science 18:1055), recently reported the production of cloned pigs using foetal fibroblasts. In their paper they also found that their current activation method for cattle did not work in pigs. These workers reported that by increasing ionomycin concentration threefold from 5 μM to 15 μM in the presence of low calcium levels they were able to activate porcine oocytes. These workers used in vitro fertilized embryos to help initiate and maintain pregnancy. Using this method these worker reported that 23% of oocytes develop parthenogenetically to blastocyst stage (a model for activation of nuclear transfer embryos). This is similar to what we and others have reported previously using, for example, electrical stimulation. Using the present method described herein we have increased parthenogenetic activation from around 15% using electrical activation to approximately 50%. Thus, the present method represents a considerable improvement over existing methods. More importantly the rate at which embryos develop to the blastocyst stage appears to be increased also by this method. (Table 2)

NT embryos can be cultured in vitro for one or more divisions. After cleavage, the NT embryo can be bisected at any suitable stage, (for example, at the 2 to 32 cell stage) using physical or chemical means (embryo splitting). Embryonic cells or blastomeres may be isolated therefrom and used in second and subsequent rounds of nuclear transfer to produce multiple NT embryos capable of development to term (serial cloning).

A second round of nuclear transfer has been used to increase the developmental competence of mouse NT embryos (Kwon & Kono (1996) Proc. Natl. Acad. Sci. USA 93:13010). The second cytoplast can be an oocyte, zygote or any other embryo.

NT embryos can be cultured in vitro for one or more divisions to assess their viability or transferred to the reproductive tract of a recipient female, or stored frozen for subsequent use by standard procedures.

The present invention may include genetic manipulation of the donor cell or karyoplast prior to transfer into the recipient cytoplast. Alternatively, or in addition, genetic manipulation may take place following NT cell production, that is genetic manipulation on the NT embryo.

The invention is suitable for use with all nuclear transfer protocols including simultaneous fusion and activation, fusion before activation, post fusion enucleation, serial nuclear transfer, etc.

The invention is suitable for all species, in particular ungulates, and most particularly in pigs.

Uses for nuclear transfer or cloning technology include: the production of large numbers of genetically identical or similar animals or clones from an individual animal for purposes of animal breeding; the production of genetically manipulated, that is, transgenic animals in which extra genetic information has been inserted or existing genetic information deleted (gene knockout); and the de-differentiation of somatic cells to produce a population of pluripotent cells which can then be differentiated to cells, tissues or organs for the purpose of cell therapy, gene therapy, organ transplantation, etc. Such cells have an advantage in that they can be autologous, that is, obtained initially from the patient and as such are not destroyed by the patient's immune system.

Thus, according to the present invention, reproduction or multiplication of mammals having specific or desired genotypes is possible. In addition, the present invention can also be used to produce animals which can be used, for example, in cell, tissue or organ transplantation, or to produce animals which express desired compounds such as therapeutic molecules, growth factors, or other medically desired peptide or protein

This invention will now be described with reference to the following non-limiting examples.

### EXAMPLES

### Example 1

### Parthenogenetic Activation of IVM Porcine Oocytes

Media

The oocyte maturation medium (OMM199a) consisted of Medium 199 (with Earle's salts, L-glutamine, 2.2 mg mL⁻¹ sodium bicarbonate and 25 μM Hepes buffer; Gibco-BRL) supplemented with 0.1 mg mL⁻¹ sodium pyruvate, 75 μg mL⁻¹ penicillin-G, 50 μg mL⁻¹ streptomycin sulfate, 10 μg mL⁻¹ ovine FSH, 5.0 μg mL⁻¹ ovine LH, 1.0 μg mL⁻¹ 17β-oestradiol, 0.5 mM cysteamine, 1.0 mM dibutyryl cAMP; 10 mg mL⁻¹ epidermal growth factor (EGF) and 25% (v/v) porcine follicular fluid (pFF). The pFF was prepared by centrifugation (2,000×g for fifteen minutes) of the material collected from antral follicles, stored at −20° C. and filtered through a sterile 0.22 μm pore filter (Millipore, Mass., USA) immediately prior to use. The culture medium consisted of NCSU-23 medium (Petters and Wells, 1993) supplemented with 4.0 mg mL⁻¹ BSA. The TALP-PVA medium (114.0 mM NaCl, 3.16 mM KCl, 0.35 mM NaH₂PO₄.2H₂O, 0.5 mM MgSO₄.6H₂O, 25 mM NaHCO₃, 2 mg/L phenol red, 0.1% PVA, 75 mg/L penicillin-G, 50 mg/L streptomycin sulfate, 4.72 mM CaCl₂.2H₂O, 10.0 mM sodium lactate, 0.10 mM sodium pyruvate) was modified by the addition of 2.0 mM caffeine-sodium benzoate, 3.0 mM calcium lactate and 0.4% BSA (mTALP-PVA).

Methods

In Vitro Maturation

The preparation of in vitro matured (IVM) oocytes was essentially as described previously (Grupen et al, (1997) Reproduction Fertility Development 9:571-575). Ovaries from slaughtered prepubertal gilts were transported to the laboratory in Dulbecco's PBS supplemented with 0.6% (v/v) of an antibiotic solution (CSL Ltd) containing penicillin (10,000 U mL⁻¹), streptomycin (10,000 μg mL⁻¹) and fungizone (25 μg mL⁻¹) and maintained at 38° C. Antral follicles (2 mM to 6 mM in diameter) were aspirated using a 21-gauge needle through which constant suction (1 L min⁻¹) was applied. The follicular contents were pooled in a collection tube. Cumulus-oocyte complexes (COCs) with at least three uniform layers of compact cumulus cells were recovered from the collected fluid, washed three times in OMM199a, transferred to 50 μl droplets (25 COCs per droplet) of OMM199a covered with mineral oil in a petrie dish (Becton Dickinson and Company, Plymouth, England) and incubated at 38.5° C. in a humidified atmosphere of 5% CO₂ in air. After twenty two hours of maturation, expanded COCs were washed once in OMM199 without dibutyryl CAMP (OMM199b), transferred to 50 μl droplets of OMM199b and incubated for a further twenty four hours. At the end of the forty six hours maturation interval, the oocytes were treated with 0.5 mg mL⁻¹ hyaluronidase for one minute and then gently aspirated with a small bore glass pipette to remove the cumulus cells. Oocytes that had extruded a polar body were washed and kept in culture medium prior to activation.

The oocyte maturation medium (OMM199a) consisted of Medium 199 (with Earle's salts, L-glutamine, 2.2 mg mL⁻¹ sodium bicarbonate and 25 μM Hepes buffer; Gibco-BRL) supplemented with 0.1 mg mL⁻¹ sodium pyruvate, 75 μg mL⁻¹ penicillin-G, 50 μg mL⁻¹ streptomycin sulfate, 10 μg mL⁻¹ ovine FSH, 5.0 μg mL⁻¹ ovine LH, 1.0 μg mL⁻¹ 17β-oestradiol, 0.5 mM cysteamine, 1.0 mM dibutyryl cAMP, 10 mg mL⁻¹ epidermal growth factor (EGF) and 25% (v/v) porcine follicular fluid (pFF). The pFF was prepared by centrifugation (2,000×g for fifteen minutes) of the material collected from antral follicles, stored at −20° C. and filtered through a sterile 0.22 μm pore filter (Millipore, Mass., USA) immediately prior to use. The culture medium consisted of NCSU-23 medium (Petters and Wells, 1993) supplemented with 4.0 mg mL⁻¹ BSA.

The experiments hereafter were conducted with oocytes rather than nuclear transfer embryos. These experiments are termed parthenogenetic, since they are done without fertilized embryos. Parthenogenetic experiments are used routinely as a model system to investigate NT, largely because of the expense and additional experimental manipulation involved with NT embryos. Parthenogenetic experiments provide very useful information for the development of NT technology, and are directly predictive of NT outcomes.

Electrical Activation

Oocytes were activated from six to forty eight hours after the start of maturation. Oocytes were removed from the culture medium and washed once and transferred to PB1 medium prior to activation. Oocytes to be pulsed were washed thoroughly in activation medium, placed in activation medium between the stainless steel electrodes (1 mM apart) of an activation chamber slide and exposed to two DC pulses (1.5 kV cm⁻¹, 60 μsec), which were applied one second apart using a BTX Electro Cell Manipulator 2001 (BTX, Inc., San Diego, Calif., USA). The oocyte activation medium contained 0.3 M mannitol, 0.1 mM CaCl₂, 0.2 mM MgSO₄ and 0.1 mg mL⁻¹ polyvinylalcohol. Oocytes were washed twice and transferred to PB1 medium immediately after the administration of pulses. Pulsed oocytes were either washed twice and returned to the culture medium oil.

Ionomycin/6-DMAP Treatment

Oocytes were activated forty six to forty eight hours after the start of maturation. Denuded oocytes that had extruded a polar body were washed and kept in modified TALP-PVA medium supplemented with 3.0 mM Ca-lactate (mTALP-PVA) for approximately one hour prior to activation. Oocytes were transferred to mTALP-PVA containing 5 μM ionomycin for five minutes. Oocytes were then washed twice and incubated in culture medium containing 2 mM 6-DMAP for three hours. The 6-DMAP treated oocytes were then washed twice and transferred to 50 μl droplets of the culture medium under mineral oil. In the ionomycin * treatment oocytes were not washed in media containing 6-DMAP. Exposure to 6-DMAP did not occur until five to ten minutes later when oocytes were incubated in NCSU-23.

TABLE 1
Effect of Ca²⁺ concentration on ionomycin + 6-DMAP
stimulated activation

| Medium | n | Percentage developing to blastocyst |
|---|---|---|
| Low Ca²⁺ (1.4 mM) | 40 | 5 |
| High Ca²⁺ (7.7 mM) | 40 | 65** |

**Sig diff (P < 0.001)

TABLE 2
Parthenogenetic development of
artificially activated IVM porcine oocytes.

Percentage developing
to blastocyst

| Treatment | 6-DMAP | n | Day 6 | Day 7 |
|---|---|---|---|---|
| Electrical | − | 45 | 11^{a} | 18^{a} |
| Ionomycin | + | 55 | 49^{c} | 51^{c} |
| Ionomycin* | + | 74 | 1^{d} | 1^{d} |

Within columns numbers with different superscripts are significantly different (P < 0.05) Superscript a, c and d in Table 2 refer to values that are statistically different.

### Example 2

### Activation of Nuclear Transfer Embryos Constructed Using IVM Oocytes as Recipient Cytoplasts

Media

The culture medium and the mTALP-PVA medium are described in Example 1. Dulbecco's phosphate buffered saline (DPBS; 136.98 mM NaCl, 2.68 mM KCl, 0.49 mM MgCl₂.6H₂O, 8.08 mM Na₂HPO₄, 1.47 mM KH₂PO₄ and 0.90 mM CaCl₂.2H₂O; pH 7.4) was supplemented with 1% foetal calf serum (FCS). Hepes-buffered MEM consisted of Minimum Essential Medium (with Earle's salts, L-glutamine and non-essential amino acids; Gibco-BRL, Grand Island, N.Y., USA) supplemented with 336 mg/L NaHCO₃, 21 mM Hepes buffer, 60 mg/L penicillin-G and 0.5% bovine serum albumin (BSA). Phosphate-buffered NCSU-23 (pNCSU-23) medium contained 127.8 mM NaCl, 4.97 mM KCl, 1.0 mM KH₂PO₄, 1.19 mM MgSO₄.7H₂O, 3.0 mM Na₂HPO₄, 5.55 mM D-glucose, 75 mg/L penicillin-G, 50 mg/L streptomycin sulfate, 1.7 mM CaCl₂, 1.0 mM L-glutamine, 7.0 mM taurine, 5.0 mM hypotaurine, 0.4% BSA and 10% FCS. Ca²⁺-free pNCSU-23 medium contained 127.8 mM NaCl, 4.97 mM KCl, 1.0 mM KH₂PO₄, 1.19 mM MgSO₄.7H₂O, 3.0 mM Na₂HPO₄, 5.55 mM D-glucose, 75 mg/L penicillin-G, 50 mg/L streptomycin sulfate, 1.0 mM L-glutamine, 7.0 mM taurine, 5.0 mM hypotaurine, 0.4% BSA and 10% FCS. The Ca²⁺-free mannitol fusion medium consisted of 0.28 M mannitol, 0.2 mM MgSO₄ and 0.01% polyvinylalcohol. Dulbecco's Modified Eagle Medium (DMEM) contained high glucose with L-glutamine, 110 mg/L sodium pyruvate and pyridoxine hydrochloride.

In Vitro Maturation

The method is described in Example 1.

Donor Cell Preparation

Primary cultures of porcine foetal fibroblast cells were grown to confluence after seven to fourteen days in DMEM supplemented with 15% FCS in a humidified atmosphere of 5% CO₂ in air at 38.5° C. The donor cells were prepared for nuclear transfer by washing confluent monolayers twice with DPBS followed by the addition of DPBS containing 0.05% trypsin. After 5 minutes of incubation at 38.5° C., DMEM+15% FCS was added to dissociated cells to stop the trypsin reaction. Dissociated cells were then pelleted by centrifugation at 300×g for 5 minutes and resuspended in DMEM+15% FCS. Dissociated cells were incubated at 5% CO₂, 39° C. for at least 0.5 hours prior to micromanipulation.

Micromanipulation

For micromanipulation, oocytes and cells were placed in a drop under oil of pNCSU-23 with 7.5 μg/ml cytochalasin B and 10% FCS. Oocytes were enucleated by removing the first polar body along with adjacent cytoplasm containing the metaphase plate using a micropipette with an inner diameter of about 20 μm. In a majority of oocytes, the metaphase plate was visible under phase contrast optics as a clear space contrasted against dark cytoplasm. Through the same hole in the zona pellucida created during enucleation, a small to medium-sized donor cell was then placed in contact with the cytoplasm of each oocyte to create a couplet. After manipulation, couplets were washed once, transferred to NCSU-23 supplemented with 10% FCS and incubated in a humidified atmosphere of 5% CO₂ in air at 38.5° C. for at least 0.5 hours before fusion.

Couplet Fusion

Prior to fusion, couplets were washed, transferred to Ca²⁺-free pNCSU-23 medium and incubated at 38° C. for at least 15 minutes. Groups of up to 10 couplets were washed thoroughly in Ca²⁺-free mannitol fusion medium and then transferred to a fusion chamber with electrodes 1 mm apart overlaid with fusion medium. Couplets were manually aligned using a 30 gauge needle so that the plane of contact between the donor and recipient cells was parallel with the electrodes. Cell fusion was induced with a single DC pulse of 150 V/mm field strength and 60 μsec duration. Couplets were also exposed to a 4.0 V AC pulse for two second immediately prior to the fusion pulse and to an AC pulse immediately after the fusion pulse, that diminished from 4.0 to 0.0 V over a two second interval. After the electric pulse was administered, the couplets were washed, transferred to Ca²⁺-free pNCSU-23 medium and incubated at 38° C. for at least fifteen minutes. Unfused couplets were exposed to the same fusion procedure a second time. Fused couplets (cybrids) were washed once, transferred to NCSU-23 supplemented with 10% FCS and incubated in a humidified atmosphere of 5% CO₂ in air at 38.5° C. for one to three hours prior to activation.

Ionomycin/6-DMAP

The method is essentially the same as that described in Example 1. One to 3 h post fusion, the fused couplets (cybrids) were washed, transferred to mTALP-PVA medium and incubated for 15 to 20 minutes prior to activation. Fused couplets were then transferred to mTALP-PVA medium containing 5 μM ionomycin for five minutes. Fused couplets were then washed twice, transferred to 50 μl droplets of culture medium containing 2 mM 6-DMAP covered with mineral oil and incubated for three hours in a humidified atmosphere of 5% CO₂ in air at 38.5° C. Activated fused couplets were then washed twice and transferred to 50 μl droplets of culture medium covered with mineral oil and cultured for either seven days to assess in vitro development or for three days prior to transfer into a synchronized recipient.

Results

The data presented in Table 3 shows that the ionomycin/6-DMAP treatment effectively activated nuclear transfer embryos reconstructed using IVM oocytes as recipient cytoplasts. A high rate of development to the blastocyst stage (21%) was achieved after seven days of culture in vitro when fused couplets (cybrids) were activated 3 hours after fusion using the ionomycin/6-DMAP treatment. Fused couplets that were not subjected to the ionomycin/6-DMAP treatment failed to form blastocysts and cleaved at a rate of only 17%, indicating that the majority of cybrids remained unactivated following fusion.

TABLE 3
Effect of the Ionomycin/6-DMAP treatment on the development of
cybrids constructed using IVM pig oocytes.

| Treatment | n | Cleaved (%) | Blastocyst (%) |
|---|---|---|---|
| Ionomycin/6-DMAP | 105 | 63 (60) | 22 (21) |
| Untreated | 76 | 13 (17) | 0 (0) |

### Example 3

### Production of Cloned Pigs Following Activation of Nuclear Transfer Embryos Constructed Using in Vivo-Derived Oocytes as Recipient Cytoplasts

Media

The culture medium, mTALP-PVA medium, DPBS, Hepes-buffered MEM, pNCSU-23 medium, Ca²⁺-free pNCSU-23 medium, Ca²⁺-free mannitol fusion medium and DMEM are described in Examples 1 and 2.

Oocyte Recovery

Freshly ovulated oocytes were flushed from the oviducts of superstimulated pig donors 48 h after hCG injection with DPBS and transported to the laboratory in Hepes-buffered MEM. They were then stripped of the attached cumulus cells by pipetting in pNCSU-23 containing 1 mg/ml hyaluronidase. Stripped oocytes were then washed and transferred to culture medium supplemented with 10% FCS and incubated in a humidified atmosphere of 5% CO₂ at 38.5° C. for 0.5 to two hours prior to micromanipulation.

Donor Cell Preparation

The method is described in Example 2.

Micromanipulation

The method is described in Example 2.

Couplet Fusion

The method is the same as that described in Example 2 except pNCSU-23 medium was used instead of Ca²⁺-free pNCSU-23 medium.

Ionomycin/6-DMAP

The method is the same as that described in Example 2 except that the fused couplets were incubated in mTALP-PVA for thirty to forty five minutes prior to activation.

Results

The data presented in Table 4 shows that the ionomycin/6-DMAP treatment effectively activated nuclear transfer embryos reconstructed using in vivo-derived oocytes as recipient cytoplasts. A high rate of development to the blastocyst stage (23%) was achieved after six days of culture in vitro when fused couplets (cybrids) were activated three hours after fusion using the ionomycin/6-DMAP treatment. Fused couplets that were not subjected to the ionomycin/6-DMAP treatment failed to form blastocysts and cleaved at a rate of only 7%, indicating that the majority of cybrids remained unactivated following fusion. Differential labelling after six days of in vitro culture showed that the nuclear transfer blastocysts had good numbers of inner cell mass (average 10) and trophectoderm cells (average 31).

TABLE 4
Development of fused couplets with or without activation using
Ionomycin followed by incubation in 6-DMAP.

| Treatment | n | Cleaved (%) | Blastocyst (%) |
|---|---|---|---|
| Ionomycin/6-DMAP | 108 | 100 (93)^{a} | 25 (23)^{a} |
| Untreated | 27 | 2 (7)^{b} | 0 (0)^{b} |

Within columns values with different superscripts are significantly different (P < 0.05)

The efficiency of the ionomycin/6-DMAP treatment was also demonstrated when reconstructed embryos were cultured for three days and subsequently transferred to synchronized recipients Table 5). Approximately 70% of constructed couplets were successfully fused. Following activation using the ionomycin/6-DMAP treatment, 90% of the fused couplets cleaved and were subsequently transferred to recipients after three days of in vitro culture. From ten transfers, five recipients were found to be pregnant at day twenty five. Two of the recipients remained pregnant to term and one live cloned piglet was obtained from each, one male and one female. This data demonstrates that the ionomycin/6-DMAP treatment efficiently activated nuclear transfer embryos constructed using in vivo-derived oocytes as recipient cytoplasts and did not compromise the capacity of the nuclear transfer embryos to develop to term.

TABLE 5
Development of nuclear transfer embryos constructed using in vivo-derived oocytes as recipient cytoplasts.

| | | | | | | | | | | | | Recipient | | |
|---|---|---|---|---|---|---|---|---|---|---|---|---|---|---|
| | Manipulation | | Fusion | | | D 3 development | | | | | | Embryos | Ultrasound | |
| Trial | Cell line | Couplets | F1¹ | F2² | Total³ | Lysed/frag | 1-cell | 2-cell | 3-cell | 4-cell | 5-cell+ | transferred⁴ | result⁵ | Outcome⁶ |
| 1 | 25.21.p12 | 99 | 41 | 23 | 64 (65) | 5 | 8 | 8 | 5 | 26 | 13 | 52 (81) | pregnant | 1 piglet |
| 2 | 25.21.p13 | 108 | 24 | 25 | 49 (45) | 7 | 2 | 6 | 3 | 13 | 18 | 40 (82) | not pregnant | returned |
| 3 | 25.21.p13/ | 103 | 52 | 25 | 77 (75) | 9 | 6 | 20 | 13 | 24 | 5 | 62 (81) | not pregnant | returned |
| | 25.15.p4 | | | | | | | | | | | | | |
| 4 | 25.15.p5 | 106 | 77 | 17 | 94 (89) | 0 | 1 | 13 | 9 | 44 | 29 | 93 (99) | not pregnant | returned |
| 5 | 25.15.p5 | 148 | 78 | 32 | 110 (74) | 1 | 3 | 16 | 20 | 47 | 23 | 106 (96) | pregnant | 1 piglet |
| 6 | 25.15.p6 | 121 | 66 | 30 | 96 (79) | 5 | 3 | 18 | 17 | 34 | 19 | 88 (92) | pregnant | aborted (44) |
| 7 | 25.15.p6 | 156 | 89 | 32 | 121 (78) | 0 | 11 | 23 | 25 | 37 | 22 | 107 (88) | pregnant | aborted (27) |
| 8 | 25.15.p6 | 131 | 44 | 39 | 83 (63) | 1 | 6 | 17 | 11 | 28 | 15 | 71 (85) | not pregnant | returned |
| 9 | 25.15.p7 | 143 | 72 | 32 | 104 (73) | 6 | 5 | 27 | 16 | 36 | 20 | 99 (95) | pregnant | aborted (28) |
| 10 | 25.15.p10 | 95 | 40 | 30 | 70 (74) | 2 | 3 | 19 | 6 | 33 | 7 | 55 (79) | not pregnant | returned |

¹First round of fusion
²Second round of fusion
³Per cent of couplets
⁴Per cent of fused embryos
⁵Ultrasounds were conducted between d 25 to d 35 after oestrus
⁶Day aborted after oestrus

Those skilled in the art will appreciate that the inventions described herein is susceptible to variations and modifications other than those specifically described. It is to be understood that the invention includes all such variations and modifications which fall within its spirit and scope. The invention also includes all of the steps, features, compositions and compounds referred to or indicated in this specification, individually or collectively, and any and all combinations of any two or more of said steps or features.

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

1. A method for the activation of nuclear transferred embryos using elevated calcium levels introduced into cells, and a maturation promoting factor (MPF) inhibitor.

2. A method according to claim 1, wherein elevated calcium levels are introduced into the embryo cells from a culture medium containing elevated calcium levels.

3. A method according to claim 2 wherein said elevated calcium levels are in the range from about 2 mM to about 12 mM.

4. A method according to claim 1 wherein elevated calcium levels are introduced into cells by a calcium ionophore or other physical or chemical means.

5. A method according to claim 1 wherein following introduction of elevated calcium levels into cells, the cells are directly and immediately followed by incubation with an MPF inhibitor

6. A method according to claim 5 wherein elevated extra cellular calcium levels are removed prior to incubation in the presence of MPF inhibitor.

7. A method according to claim 1 wherein the embryo is a single cell embryo, or an activated multicellular embryo.

8. A method according to claim 1 wherein the embryo is incubated in an embryonic culture medium to give developmentally programmed cell division.

9. A method according to claim 1 wherein the embryo is ungulate embryo.

10. A method according to claim 9 wherein the ungulate embryo is a pig embryo, cattle or sheep embryo.

11. An animal produced from an embryo according to claim 1.

12. A method for activating a nuclear transfer embryo of a mammal, comprising incubating the embryo in an embryo culture medium including an elevated calcium level and a divalent cation ionophore for a period sufficient to introduce calcium into the embryo to give a calcium loaded embryo, immediately removing the ionophore by washing the embryo in the presence of an MPF inhibitor, and thereafter incubating the calcium loaded embryo with an MPF inhibitor to give an activated nuclear transfer embryo.

13. A method according to claim 12, wherein elevated calcium levels are introduced into the embryo from a culture medium containing elevated calcium levels.

14. A method according to claim 12 wherein said elevated calcium levels are in the range from about 2 mM to about 12 mM.

15. A method according to claim 12 wherein elevated calcium levels are introduced into cells by a calcium ionophore or other physical or chemical means.

16. A method according to claim 12 wherein following introduction of elevated calcium levels into cells, the cells are directly and immediately followed by incubation with an MPF inhibitor.

17. A method according to claim 12 wherein elevated extra cellular calcium levels are removed prior to incubation in the presence of MPF inhibitor.

18. A method according to claim 12 wherein the embryo is a single cell embryo, or an activated multicellular embryo.

19. A method according to claim 12 wherein the embryo is incubated in an embryonic culture medium to give developmentally programmed cell division.

20. A method according to claim 12 wherein the embryo is ungulate embryo.

21. A method according to claim 20 wherein the ungulate embryo is a pig embryo, cattle or sheep embryo.

22. An animal produced from an embryo according to claim 1.
